(19) United States
(12) Patent Application Publication
(10) Pub. No.: US 2022/0168547 A1
(43) Pub. Date: Jun. 2, 2022

(54) Catheter Delivery Device to Facilitate Blood Draw

(71) Applicant: Becton, Dickinson and Company, Franklin Lakes, NJ (US)

(72) Inventors: Yiping Ma, Layton, UT (US); Jonathan Karl Burkholz, Salt Lake City, UT (US)

(21) Appl. No.: 17/539,711

(22) Filed: Dec. 1, 2021

Related U.S. Application Data
(60) Provisional application No. 63/120,071, filed on Dec. 1, 2020.

(51) Int. Cl. A61M 25/06

(52) U.S. Cl. CPC A61M 25/0606

(57) ABSTRACT

A delivery device for delivering a catheter into an intravenous catheter assembly may include a first catheter, which may include a first portion, a second portion distal to the first portion, and a transition portion disposed between the first portion and the second portion. The first portion may include a first inner diameter along a length of the first portion and a first outer diameter along the length of the first portion. The second portion may include a second inner diameter and a second outer diameter along a length of the second portion. The second inner diameter and the second outer diameter may be less than the first inner diameter and the first outer diameter, respectively. The transition portion may be disposed distal to the distal end of the housing when the first catheter is advanced.

### CROSS-REFERENCE TO RELATED APPLICATION

The present application claims priority to U.S. Provisional Application Ser. No. 63/120,071, entitled “Catheter Delivery Device to Facilitate Blood Draw”, filed Dec. 1, 2020, the entire disclosure of which is hereby incorporated by reference in its entirety.

### BACKGROUND

Infusion therapy, a common healthcare procedure, may be facilitated by a vascular access device. Hospitalized, home care, and other patients receive fluids, pharmaceuticals, and blood products via a vascular access device inserted into the vascular system. Blood withdrawal is another common healthcare procedure that may be facilitated by a vascular access device.

A vascular access device may access a peripheral or central vasculature of a patient. A vascular access device may be indwelling for short term (days), moderate term (weeks), or long term (months to years). A vascular access device may be used for continuous infusion therapy or for intermittent therapy.

A common type vascular access device is an over-the-needle peripheral intravenous catheter (PIVC). As its name implies, the “over-the-needle” PIVC may be mounted over an introducer needle having a sharp distal tip. The sharp distal tip may be used to pierce skin and the vasculature of the patient. Insertion of the PIVC into the vasculature may follow the piercing of the vasculature by the needle. The needle and the PIVC are generally inserted at a shallow angle through the skin into the vasculature of the patient with a bevel of the needle facing away from the skin of the patient. Once placement of the needle within the vasculature has been confirmed, the clinician may withdraw the needle, leaving the PIVC in place for future fluid infusion and/or blood withdrawal.

Currently, there may be several limitations to the use of a PIVC for fluid infusion or blood draw. The PIVC or vein may narrow, collapse, or clog with time, leading to failure of the PIVC. Also, blood extracted from PIVCs may often need to be discarded due to concerns regarding sample quality, which may result in an unusable sample and a need to repeat blood collection. Further, use of a PIVC to draw blood can be slow and somewhat inefficient, particularly when the patient has difficult intravenous access or veins that are not readily accessed by the clinician.

The subject matter claimed herein is not limited to embodiments that solve any disadvantages or that operate only in environments such as those described above. Rather, this background is only provided to illustrate one example technology area where some implementations described herein may be practiced.

### SUMMARY OF THE INVENTION

The present disclosure relates generally to vascular access systems, devices, and methods. More particularly, in some embodiments, the present disclosure relates to systems, devices, and methods for placing a first catheter within a second catheter and/or a vein of a patient. In some embodiments, the second catheter may include an indwelling peripheral intravenous catheter (PIVC). In some embodiments, the first catheter may allow a user to draw a blood sample or infuse fluid through the second catheter when the second catheter is no longer functional or less effective due to, for example, debris build up on a distal end of the second catheter or collapse of the second catheter. Thus, in some embodiments, the first catheter may reduce a number of needle sticks that a patient experiences as the second catheter may be replaced less frequently.

In some embodiments, a delivery device for delivering the first catheter into an intravenous catheter assembly and/or the vein may include a housing, which may include a housing, comprising a distal end, a proximal end, and a slot. In some embodiments, the first catheter may include a proximal end and a distal end. In some embodiments, the first catheter may include a first portion, a second portion, and a transition portion disposed between the first portion and the second portion.

In some embodiments, the first portion may include a first inner diameter along a length of the first portion and a first outer diameter along the length of the first portion. In some embodiments, the second portion may be disposed distal to the first portion. In some embodiments, the second portion may include a second inner diameter and a second outer diameter along a length of the second portion. In some embodiments, the second inner diameter is less than the first inner diameter. In some embodiments, the second outer diameter may be less than the first outer diameter.

In some embodiments, the delivery device may include an advancement tab moveable along the slot to advance the first catheter in a distal direction. In some embodiments, the transition portion may be disposed distal to the distal end of the housing when the first catheter is advanced.

In some embodiments, a distal end of the second portion may form a distal end of the first catheter. In some embodiments, the transition portion may be immediately between the first portion and the second portion. In some embodiments, at least a portion of an outer surface of the transition portion may be tapered or stepped. In some embodiments, at least a portion of an inner surface of the transition portion may be tapered or stepped.

In some embodiments, the first catheter may include a third portion and another transition portion disposed between the third portion and the second portion. In some embodiments, the third portion may be disposed distal to the second portion. In some embodiments, the third portion may include a third inner diameter along a length of the third portion and a third outer diameter along a length of the third portion. In some embodiments, the third inner diameter may be less than the second inner diameter. In some embodiments, the third outer diameter may be less than the second outer diameter.

In some embodiments, at least a portion of an outer surface of the other transition may be tapered or stepped. In some embodiments, at least a portion of an inner surface of the other transition may be tapered or stepped. In some embodiments, a distal end of the third portion may form a distal end of the first catheter. In some embodiments, the other transition portion may be immediately between the third portion and the second portion. In some embodiments, the first portion may include the proximal end of the first catheter.

In some embodiments, the proximal end of the first catheter may be coupled to an extension tube. In some embodiments, the first catheter or the extension tube extend through the proximal end of the housing. In some embodiments, a proximal end of the extension tube may be configured to couple to a blood collection device.

In some embodiments, the delivery device may be coupled to a catheter assembly. In some embodiments, the catheter assembly may include a catheter adapter and a second catheter secured to the catheter adapter and extending distally from the catheter adapter. In some embodiments, the catheter adapter may include a distal end, a proximal end, a lumen extending between the distal end and the proximal end.

In some embodiments, the transition portion may be disposed within the catheter adapter when the first catheter is advanced. In some embodiments, a distal end of the second portion may form a distal end of the first catheter, and the distal end of the second portion may be disposed within the second catheter when the first catheter is advanced. In some embodiments, an inner surface of the second catheter may include a distal taper. In some embodiments, the distal end of the second portion may contact the distal taper when the first catheter is advanced. In some embodiments, the transition portion may be immediately between the first portion and the second portion.

In some embodiments, the first catheter may include the third portion and the other transition portion. In some embodiments, the other transition portion may be disposed within the second catheter when the first catheter is advanced. In some embodiments, an inner surface of the second catheter may include the distal taper, and the other transition portion may contact the distal taper when the first catheter is advanced.

In some embodiments, the other transition portion may be immediately between the third portion and the second portion. In some embodiments, a distal end of the third portion may form a distal end of the first catheter. In some embodiments, the distal end of the third portion may be disposed distal to the second catheter when the first catheter is advanced.

In some embodiments, a method may include coupling the delivery device to the catheter assembly. In some embodiments, the advancement tab may be moveable along the slot to advance the first catheter in a distal direction, and the transition portion is disposed distal to the distal end of the housing when the first catheter is advanced. In some embodiments, the method may include advancing the first catheter in the distal direction. In some embodiments, in response to advancing the first catheter in the distal direction, the transition portion may be disposed within the catheter adapter. In these and other embodiments, the first catheter further may include the third portion and the other transition portion disposed distal to the first portion and the second portion.

It is to be understood that both the foregoing general description and the following detailed description are examples and explanatory and are not restrictive of the invention, as claimed. It should be understood that the various embodiments are not limited to the arrangements and instrumentality shown in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE DRAWINGS

Example embodiments will be described and explained with additional specificity and detail through the use of the accompanying drawings in which:

FIG. 1A is an upper perspective view of an example delivery device, illustrating an example catheter in an example fully retracted position, according to some embodiments;

FIG. 1B is a cross-sectional view of the delivery device, illustrating the catheter in the fully retracted position, according to some embodiments;

FIG. 1C is an upper perspective view of an example catheter system that includes the delivery device, illustrating the catheter in the fully retracted position, according to some embodiments;

FIG. 1D is a cross-sectional view of a distal portion of the catheter system, illustrating the catheter in an example fully advanced position, according to some embodiments;

FIG. 2 is another cross-sectional view of a distal portion of the catheter system, illustrating the catheter in the fully advanced position, according to some embodiments;

FIG. 3 is another cross-sectional view of a distal portion of the catheter system, illustrating the catheter in the fully advanced position, according to some embodiments;

FIG. 4 is another cross-sectional view of a distal portion of the catheter system, illustrating the catheter in the fully advanced position, according to some embodiments;

FIG. 5A is a bar graph illustrating average fluid resistance, according to some embodiments; and

FIG. 5B is a bar graph illustrating average of blood collection rate ratio and average of maximum shear ratio, according to some embodiments.

### DESCRIPTION OF EMBODIMENTS

The present disclosure relates generally to vascular access systems, devices, and methods. More particularly, in some embodiments, the present disclosure relates to systems, devices, and methods for placing a first catheter within a second catheter and/or a vein of a patient. In some embodiments, the second catheter may include an indwelling PIVC.

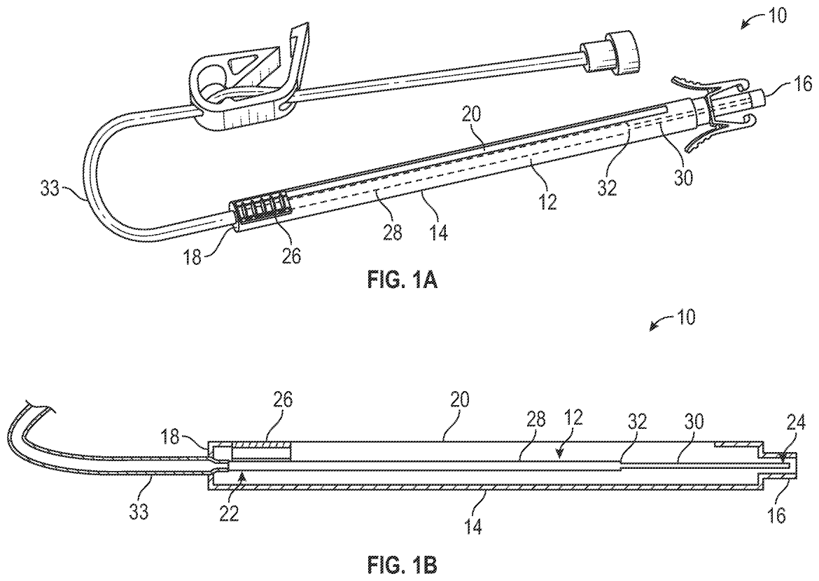

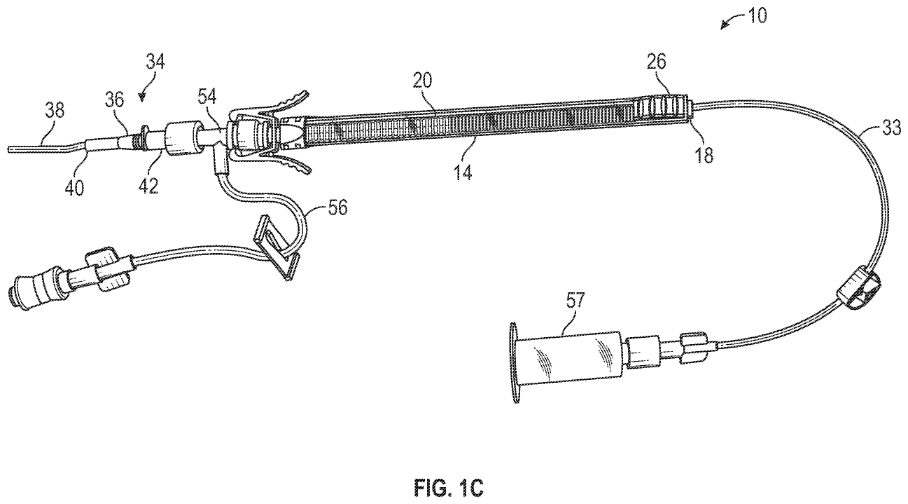

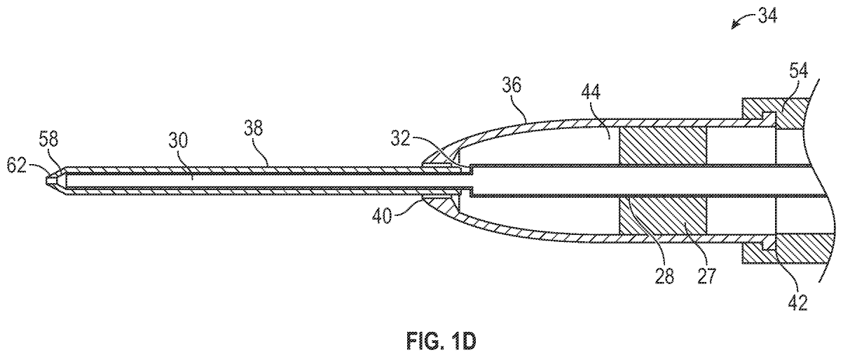

Referring now to FIG. 1A-1B, in some embodiments, a delivery device 10 for delivering a first catheter 12 into an intravenous catheter assembly and/or the vein may include a housing 14, which may include a distal end 16, a proximal end 18, and a slot 20. In some embodiments, the delivery device 10 may include the first catheter 12, which may include a proximal end 22 and a distal end 24.

In some embodiments, the delivery device 10 may include an advancement tab 26 moveable along the slot 20 to advance the first catheter 12 in a distal direction and/or retract the first catheter 12 in a proximal direction. In some embodiments, the advancement tab 26 may be coupled to the first catheter 12. In some embodiments, the first catheter 12 may be advanced distally through a septum 27, which may be secured within the lumen 44 of the catheter adapter 36.

In some embodiments, the first catheter 12 may include a first portion 28 and a second portion 30. In some embodiments, the first portion 28 may include a first inner diameter along a length of the first portion 28 and a first outer diameter along the length of the first portion 28. In some embodiments, the length of the first portion 28 may include an entire length of the first portion 28. In some embodiments, the first portion 28 may include a uniform outer diameter and a uniform inner diameter along the entire length of the first portion 28.

In some embodiments, the second portion 30 may be disposed distal to the first portion 28. In some embodiments, the second portion 30 may include a second inner diameter and a second outer diameter along a length of the second portion 30. In some embodiments, the length of the second portion 30 may include an entire length of the second portion 30. In some embodiments, the second portion 30 may include a uniform outer diameter and a uniform inner diameter along the entire length of the second portion 30. In some embodiments, the second inner diameter may be less than the first inner diameter. In some embodiments, the second outer diameter may be less than the first outer diameter.

In some embodiments, the first catheter 12 may include a transition portion 32 disposed between the first portion 28 and the second portion 30. In some embodiments, the transition portion 32 may be immediately between the first portion 28 and the second portion 30. In some embodiments, at least a portion of an outer surface of the transition portion 32 may be tapered or stepped. In some embodiments, at least a portion of an inner surface of the transition portion 32 may be tapered or stepped.

In some embodiments, the transition portion 32 may be disposed distal to the distal end of the housing 14 when the first catheter 12 is advanced. In some embodiments, the transition portion 32 may be disposed distal to the distal end of the housing 14 when the first catheter 12 is fully advanced, which may occur in response to the advancement tab 26 being moved to a distal end of the slot 20 and/or contacting a stop surface. In some embodiments, a distal end of the second portion 30 may form a distal end or distal-most surface of the first catheter 12. In some embodiments, the first portion 28 may include the proximal end 22 of the first catheter 12, which may be coupled to the advancement tab 26.

In some embodiments, the proximal end 22 of the first catheter 12 may be coupled to an extension tube 33. In some embodiments, the first catheter 12 or the extension tube 33 extend through the proximal end 18 of the housing 14. In some embodiments, a proximal end of the extension tube 50 may be coupled to a luer connector or another suitable connector, which may be coupled to a blood collection device.

Referring now to FIG. 1C, in some embodiments, the delivery device 10 may be coupled to a catheter assembly 34. In some embodiments, the distal end 16 of the housing 14 may include a luer adapter, such as a male or female luer adapter, or another suitable connector, which may facilitate coupling to the catheter assembly 34. In some embodiments, the catheter assembly 34 may include a catheter adapter 36 and a second catheter 38 secured to the catheter adapter 36 and extending distally from the catheter adapter 36. In some embodiments, the catheter adapter 36 may include a distal end 40, a proximal end 42, and a lumen 44 (see, for example, FIG. 1D) extending between the distal end 40 and the proximal end 42.

In some embodiments, the first catheter 12 may provide structural support to the second catheter 38. In some embodiments, the first catheter 12 may allow the user to draw a blood sample or infuse fluid through the second catheter 38 when the second catheter 38 is no longer functional or safe due to, for example, debris build up on a distal tip of the second catheter 38 and/or collapse of the second catheter 38. Thus, in some embodiments, the first catheter 12 may reduce a number of needle sticks that the patient experiences because the second catheter 38 may be replaced less frequently. In some embodiments, delivery of the first catheter 12 into the catheter assembly 34 and/or the vasculature of the patient may reduce blood hemolysis and a risk of kinking of the second catheter 38.

In some embodiments, the transition portion 32 may be disposed within the catheter adapter 36 when the first catheter 12 is advanced, such as, for example, fully advanced. In some embodiments, a distal end of the second portion 30 may form a distal end or distal-most surface of the first catheter 12, and the distal end of the second portion 30 may be disposed within the second catheter 38 when the first catheter 12 is advanced, such as, for example, fully advanced. In some embodiments, the transition portion 32 may be immediately between the first portion 28 and the second portion 30.

In some embodiments, the delivery device 10 may be directly coupled to the catheter adapter 36. In other embodiments, the delivery device 10 may be coupled to the proximal end 42 of the catheter adapter 36 via a connector 54, which may be disposed between the delivery device 10 and the catheter adapter 36. In some embodiments, another extension tube 56 may be integrated with a port of the connector 54.

In some embodiments, the catheter assembly 34 may include or correspond to any suitable catheter assembly, such as, for example, the BD NEXIVA™ Closed IV Catheter system, the BD CATHENA™ Catheter system, the BD VENFLON™ Pro Safely Shielded IV Catheter system, the BD NEOFLON™ IV Cannula system, the BD INSYTE™ AUTOGUARD™ BC Shielded IV Catheter system, or another suitable catheter assembly. In some embodiments, the catheter adapter 36 may include an integrated extension tube, which may include a proximal end coupled to a blood collection device.

In some embodiments, the proximal end 22 of the first catheter 12 may be coupled to the extension tube 33. In some embodiments, the first catheter 12 or the extension tube 33 may extend through the proximal end 18 of the housing 14. In some embodiments, a proximal end of the extension tube 33 may be coupled to a luer connector or another suitable connector, which may be coupled to the blood collection device.

In some embodiments, the blood collection device may include any suitable type of blood collection device. In some embodiments, the blood collection device may include a reservoir. In some embodiments, the blood collection device may include a vacuum tube, test tube, or syringe. In some embodiments, the blood collection device may include an adapter 57, which may be configured to hold a test tube or syringe. In some embodiments, the blood collection device 57 may move distally as the first catheter 12 is advanced and/or proximally as the first catheter 12 is retracted.

Referring now to FIG. 1D, in some embodiments, an inner surface of the second catheter 38 may include a distal taper 58. In some embodiments, to provide support to the second catheter 38, the distal end of the second portion 30 may contact the distal taper 58 when the first catheter 12 is advanced, such as, for example, fully advanced. In some embodiments, the distal end of the second portion 30 may be spaced apart and proximal to the distal taper 58 when the first catheter 12 is advanced, such as, for example, fully advanced. In some embodiments, the first catheter 12 may not extend into or through a distal opening 62, which may improve blood flow rates and/or reduce a risk of contamination of collected blood samples. In some embodiments, an outer diameter of the distal end of the second portion 30 may be greater than a diameter of the distal opening 62 such that the first catheter 12 is prevented from moving distally through the distal opening 62.

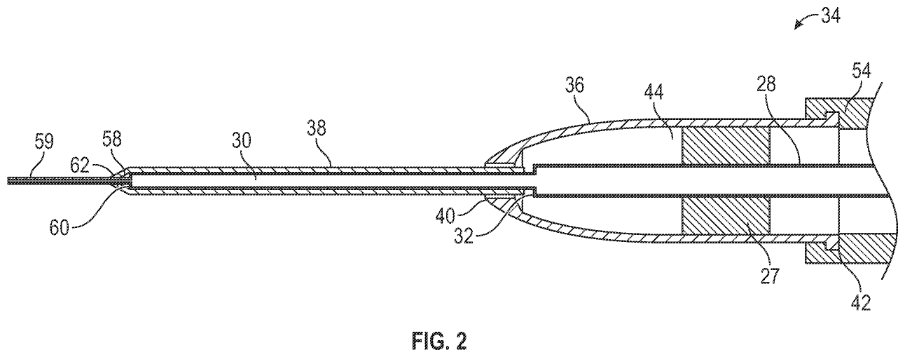

Referring now to FIG. 2, in some embodiments, the first catheter 12 may include a third portion 59, which may be disposed distal to the second portion 30. In some embodiments, the third portion 59 may include a third inner diameter along a length of the third portion and a third outer diameter along a length of the third portion. In some embodiments, the length of the third portion 59 may include an entire length of the third portion 59. In some embodiments, the third portion 59 may include a uniform outer diameter and a uniform inner diameter along the entire length of the third portion 59. In some embodiments, the third inner diameter may be less than the second inner diameter. In some embodiments, the third outer diameter may be less than the second outer diameter.

In some embodiments, the first catheter 12 may include another transition portion 60. In some embodiments, at least a portion of an outer surface of the other transition portion 60 may be tapered or stepped. In some embodiments, at least a portion of an inner surface of the other transition portion 60 may be tapered or stepped. In some embodiments, a distal end of the third portion 59 may form a distal end or distal-most surface of the first catheter 12. In some embodiments, the other transition portion 60 may be immediately between the third portion 59 and the second portion 30.

In some embodiments, the other transition portion 60 may be disposed within the second catheter 38 when the first catheter 12 is advanced, such as, for example, fully advanced. In some embodiments, to provide support to the second catheter 38, an inner surface of the second catheter 38 may include the distal taper 58, and the other transition portion 60 may contact the distal taper 58 when the first catheter 12 is advanced, such as, for example, fully advanced. In some embodiments, the other transition portion 60 may be spaced apart and proximal to the distal taper 58 when the first catheter 12 is advanced, such as, for example, fully advanced.

In some embodiments, the other transition portion 60 may be immediately between the third portion 59 and the second portion 30. In some embodiments, a distal end of the third portion 59 may form a distal end or distal-most surface of the first catheter 12. In some embodiments, the distal end of the third portion 59 may be disposed distal to the second catheter 38 when the first catheter 12 is advanced, such as, for example, fully advanced.

In some embodiments, the transition portion 32 and/or the other transition portion 60 may decrease a blood collection time because the first catheter 12 may transition to a larger inner diameter in wider portions of the catheter assembly 34, allowing blood to flow through the first catheter 12 more rapidly. In some embodiments, the delivery device 10 may improve a blood collection rate without increasing a risk of hemolysis.

In some embodiments, the outer diameter of the first portion 28 may be approximately equal to or slightly less than a diameter of the lumen 44 of the catheter adapter 36. In some embodiments, the outer diameter of the second portion 30 may be approximately equal to or slightly less than an inner diameter of the second catheter 38 proximal to the distal taper 58. In some embodiments, the outer diameter of the third portion 59 may be approximately equal to or slightly less than a diameter of a distal opening 62 of the second catheter 38, which may be aligned with a central axis of the second catheter 38.

In some embodiments, the first catheter 12 may include any number of transition portions, which may be similar to the transition portion 32 and/or the other transition portion 60. In some embodiments, a number of transition portions of the first catheter 12 may correspond to a number of areas of widening of a pathway of the first catheter 12 through the catheter assembly 34.

In some embodiments, the first catheter 12 may include tubing. In some embodiments, the tubing may be co-extruded to provide various structural improvements (layers, axial stripes, etc.). In some embodiments, the tubing may be constructed of polyimide, latex, polyurethane, nylon, polyethylene, or another suitable material. In some embodiments, one or more of the first portion 28, the second portion 30, and the third portion 59 may be cylindrical.

In some embodiments, distal tip of the first catheter 12 may include a tapered tip, a chamfered tip, or a blunt tip. In some embodiments, the distal tip of the first catheter 12 may be disposed proximate and distal to the third portion 59 or the second portion 30. In some embodiments, the distal end 24 of the first catheter 12 may include multiple side holes or fenestrations, which may provide blood collection flow entrances. In these and other embodiments, a seal or septum may be disposed within the distal end 24 of the first catheter 12, which may prevent a thrombus from entering the distal opening 62 of the first catheter 12.

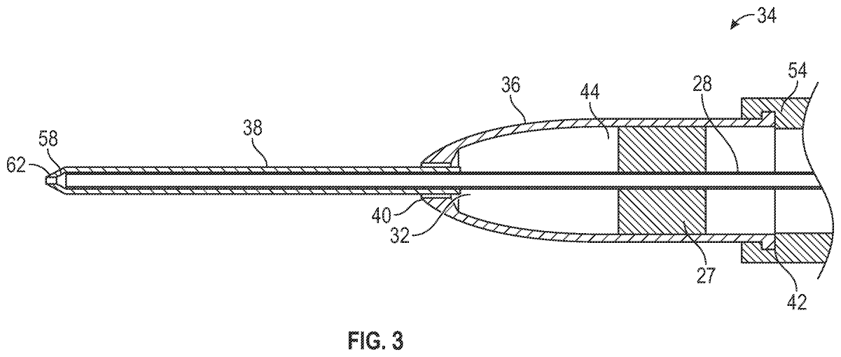

Referring now to FIG. 3, in some embodiments, the first catheter 12 may not include the second portion 30 and the third portion 59, and the first portion 28 may extend into the second catheter 38 and/or contact the distal taper 58 when the first catheter 12 is advanced, such as, for example, fully advanced. In these embodiments, an outer diameter of the first portion 28 may be less than the inner diameter of the second catheter 38. In these embodiments, the outer diameter of the distal end of the first portion 28 may be greater than the diameter of the distal opening 62 such that the first catheter 12 is prevented from moving distally through the distal opening 62.

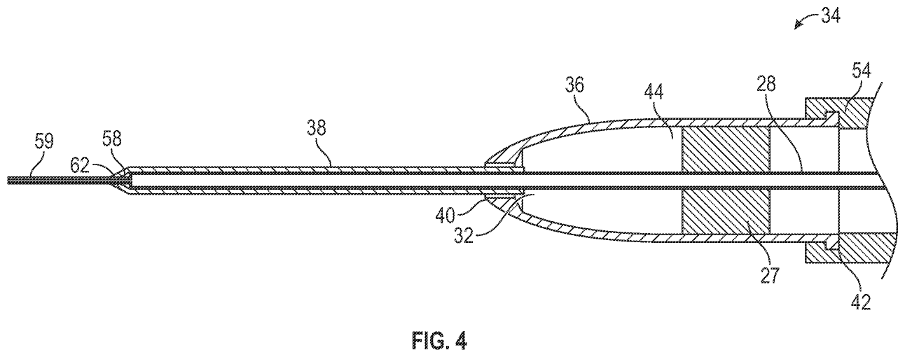

Referring now to FIG. 4, in some embodiments, the first catheter 12 may not include the second portion 30, and the first portion 28 may extend into the second catheter 38 and/or contact the distal taper 58 when the first catheter 12 is advanced, such as, for example, fully advanced. In these embodiments, an outer diameter of the first portion 28 may be less than the inner diameter of the second catheter 38. In these embodiments, the outer diameter of the distal end of the first portion 28 may be greater than the diameter of the distal opening 62 such that the first portion 28 is prevented from moving distally through the distal opening 62. In some embodiments, the third portion 59 may extend through the distal opening 62 when the first catheter 12 is advanced or fully advanced. In some embodiments, a transition portion between the first portion 28 and the third portion 59 may be similar or identical to the transition portion 32 and/or the other transition portion 60.

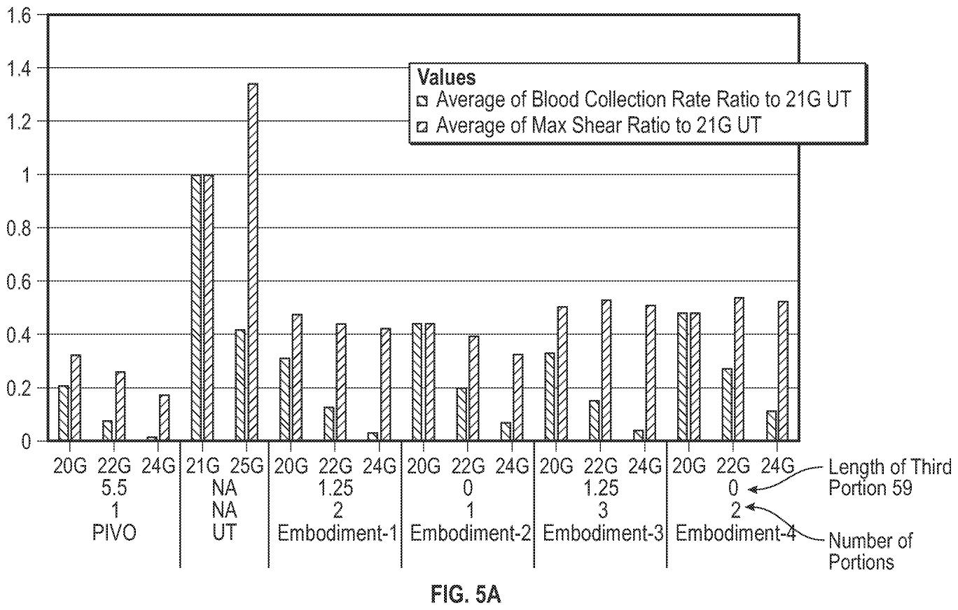

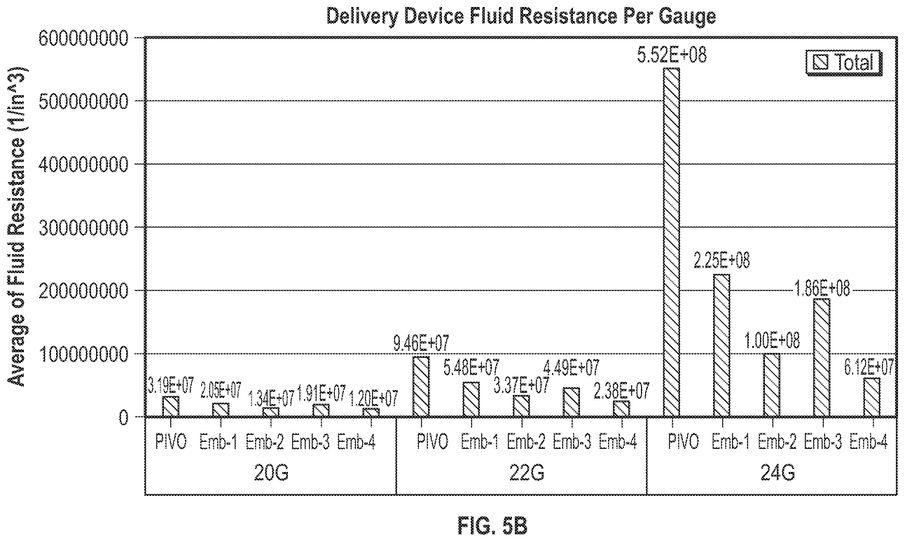

Referring now to FIG. 5A, blood draws are frequently performed with blood collection sets, such as, for example, the BD VACUTAINER® ULTRATOUCH™ push button blood collection set (“UT”), available from Becton Dickinson & Company of Franklin Lakes, N.J. UT is often relied on as the gold standard for blood collection. A common problem with instead using a peripheral intravenous catheter to draw blood from a patient is that as blood is drawn into a syringe or BD VACUTAINER® blood collection tube, red blood cells are in a high shear stress state and susceptible to hemolysis due to a high pressure differential between the vein and the syringe or BD VACUTAINER® blood collection tube. Hemolysis may result in rejection and discard of a blood sample. The high-pressure differential can also result in catheter tip collapse, vein collapse, or other complications.

In some embodiments, the first portion 28, the second portion 30, and/or the third portion 59 (see, for example, FIGS. 1A-1D and 2) may facilitate blood draw through a PIVC. As illustrated in FIG. 5A, use of the delivery device 10, according to various embodiments, may result in a 50% to 100% increase in flow rate. Further, a maximum shear stress on red blood cells is less for each of “PIVO,” “Embodiment-1,” “Embodiment-2,” “Embodiment-3,” and “Embodiment-4” than for the 21G BD VACUTAINER® ULTRATOUCH™ push button blood collection set (“UT”). “PIVO” refers to the PIVO™ device (available from VelanoVascular™ of San Francisco, Calif.) coupled to a catheter assembly, such as, for example, the catheter assembly 34. “Embodiment-1,” “Embodiment-2,” “Embodiment-3,” and “Embodiment-4” refer to embodiments of the delivery device 10, with a varying number of portions (first portion 28, second portion 30, and/or third portion 59) and a varying third portion 59 length, and coupled to the catheter assembly, such as, for example, the catheter assembly 34. The catheter assembly includes a first catheter 12 that is 20G, 22G, or 24G, according to the embodiments of FIG. 5A. As illustrated in FIG. 3A, the blood collection rate is slow for “PIVO” compared to 21G “UT”. FIG. 5A illustrates the blood collection rate is improved for “Embodiment-1”, “Embodiment-2”, “Embodiment-3” and “Embodiment-4” compared to “PIVO” and the average of max shear ratio to 21G “UT” is still less than 1.

“Embodiment-1” includes the first portion 28 with same dimensions as the second portion 30 (i.e. combined as one section, see for example, FIG. 4). “Embodiment-1” also includes the third portion 59 that protrudes through the distal opening 62. “Embodiment-2” is the same as “Embodiment-1”, except that “Embodiment-2” does not include the third portion 59 (see, for example, FIG. 3). The delivery device 10 does not protrude through the distal opening 62 for “Embodiment-2”. “Embodiment-3” includes the first portion 28, the second portion 30, and the third portion 59. “Embodiment-4” includes the first portion 28 within the catheter adapter 36 and the second portion 30 extending into the second catheter 38 but does not include the third portion 59. The delivery device 10 does not protrude through the distal opening 62 for “Embodiment-4”.

Referring now to FIG. 5B, fluid flow in a tube can be analyzed using Poiseuille's equation:

$$Q = \frac{π {D}^{4} Δ P}{128 μ L} = \frac{Δ P}{c {R}_{f}}$$

where P is a change in pressure gradient, D and L are the diameter and length of a fluid path through the tube,

$$c ( a fluid dependent constant ) = 128 μ / π$$

and R_{f}(the fluid resistance) is:

$${R}_{f} = \frac{L}{{D}^{4}}$$

For a fluid path with multiple portions of tube with lengths (L1, L2, L3) and diameters of (D1, D2, D3), the fluid resistance is:

$${R}_{f} = \frac{L 1}{D {1}^{4}} + \frac{L 2}{D {2}^{4}} + \frac{L 3}{D {3}^{4}}$$

In some embodiments, L1 and D1 may correspond to the first portion 28, L2 and D2 may correspond to the second portion 30, L3 and D3 may correspond to the third portion 59, and the fluid path may correspond to the fluid path through the first catheter 12. “PIVO,” “Embodiment-1,” “Embodiment-2,” “Embodiment-3,” and “Embodiment-4” refer to designs of the delivery device 10, with varying number of portions and third portion lengths, and coupled to a catheter assembly, such as, for example, the catheter assembly 34. In further detail, the (length of third portion 59 (inches), number of portions) of PIVO, Embodiment-1, Embodiment-2, and Embodiment-3 are (5.5, 1), (1.25, 2), (0, 1), (1.25, 3), and (0, 2), the same as in FIG. 5A. The catheter assembly includes a first catheter 12 that is 20G, 22G, or 24G, according to the embodiments of FIG. 5B. “Emb-1”, “Emb-2”, “Emb-3”, and “Emb-4” in FIG. 5B correspond to “Embodiment-1”, “Embodiment-2”, “Embodiment-3”, and “Embodiment-4”. The following table illustrates the fluid resistance, according to some embodiments:

| | Fluid resistance (1/in\hat{ }3) | |
|---|---|---|
| | Max | Min |
| 20 G | 2.90E+07 | 4.40E+06 |
| 22 G | 8.60E+07 | 4.40E+06 |
| 24 G | 5.00E+08 | 4.40E+06 |

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present disclosure have been described in detail, it should be understood that the various changes, substitutions, and alterations could be made hereto without departing from the spirit and scope of the disclosure.

What is claimed is:

1. A delivery device for delivering a catheter into an intravenous catheter assembly, comprising:
a housing, comprising a distal end, a proximal end, and a slot;
a first catheter, comprising a proximal end and a distal end, wherein the first catheter comprises a first portion, a second portion, and a transition portion disposed between the first portion and the second portion, wherein the first portion includes a first inner diameter along a length of the first portion and a first outer diameter along the length of the first portion, wherein the second portion is disposed distal to the first portion, wherein the second portion includes a second inner diameter and a second outer diameter along a length of the second portion, wherein the second inner diameter is less than the first inner diameter, wherein the second outer diameter is less than the first outer diameter; and
an advancement tab moveable along the slot to advance the first catheter in a distal direction, wherein the transition portion is disposed distal to the distal end of the housing when the first catheter is advanced.

2. The delivery device of claim 1, wherein a distal end of the second portion forms a distal end of the first catheter.

3. The delivery device of claim 1, wherein the transition portion is immediately between the first portion and the second portion.

4. The delivery device of claim 1, wherein at least a portion of an outer surface of the transition portion is tapered or stepped, wherein at least a portion of an inner surface of the transition portion is tapered or stepped.

5. The delivery device of claim 1, wherein the first catheter further comprises a third portion and another transition portion disposed between the third portion and the second portion, wherein the third portion is disposed distal to the second portion, wherein the third portion includes a third inner diameter along a length of the third portion and a third outer diameter along a length of the third portion, wherein the third inner diameter is less than the second inner diameter, wherein the third outer diameter is less than the second outer diameter.

6. The delivery device of claim 5, wherein at least a portion of an outer surface of the other transition is tapered or stepped, wherein at least a portion of an inner surface of the other transition is tapered or stepped.

7. The delivery device of claim 5, wherein a distal end of the third portion forms a distal end of the first catheter.

8. The delivery device of claim 5, wherein the other transition portion is immediately between the third portion and the second portion.

9. The delivery device of claim 1, wherein the first portion comprises the proximal end of the first catheter.

10. The delivery device of claim 1, wherein the proximal end of the first catheter is coupled to an extension tube, wherein the first catheter or the extension tube extend through the proximal end of the housing, wherein a proximal end of the extension tube is configured to couple to a blood collection device.

11. An intravenous catheter system, comprising:
a delivery device, comprising:
a housing, comprising a distal end, a proximal end, and a slot;
a first catheter, comprising a proximal end and a distal end, wherein the first catheter comprises a first portion, a second portion, and a transition portion disposed between the first portion and the second portion, wherein the first portion includes a first inner diameter along a length of the first portion and a first outer diameter along the length of the first portion, wherein the second portion is disposed distal to the first portion, wherein the second portion includes a second inner diameter and a second outer diameter along a length of the second portion, wherein the second inner diameter is less than the first inner diameter, wherein the second outer diameter is less than the first outer diameter; and
an advancement tab moveable along the slot to advance the first catheter in a distal direction; and
a catheter assembly, comprising:
a catheter adapter, comprising a distal end, a proximal end, a lumen extending between the distal end and the proximal end; and
a second catheter secured to the catheter adapter and extending distally from the catheter adapter, wherein the transition portion is disposed within the catheter adapter when the first catheter is advanced.

12. The intravenous catheter system of claim 11, wherein a distal end of the second portion forms a distal end of the first catheter, wherein the distal end of the second portion is disposed within the second catheter when the first catheter is advanced.

13. The intravenous catheter system of claim 11, wherein an inner surface of the second catheter comprises a distal taper, wherein the distal end of the second portion contacts the distal taper when the first catheter is advanced.

14. The intravenous catheter system of claim 11, wherein the second catheter comprises a distal opening, wherein an outer diameter of the second portion is greater than a diameter of the distal opening to prevent movement of the first catheter through the distal opening.

15. The intravenous catheter system of claim 11, wherein the first catheter further comprises a third portion and another transition portion disposed distal to the first portion and the second portion, wherein the third portion includes a third inner diameter along a length of the third portion and a third outer diameter along a length of the third portion, wherein the third inner diameter is less than the second inner diameter, wherein the third outer diameter is less than the second outer diameter, wherein the other transition portion is disposed within the second catheter when the first catheter is advanced.

16. The intravenous catheter system of claim 15, wherein an inner surface of the second catheter comprises a distal taper, wherein the other transition portion contacts the distal taper when the first catheter is advanced.

17. The intravenous catheter system of claim 15, wherein the other transition portion is immediately between the third portion and the second portion.

18. The intravenous catheter system of claim 15, wherein a distal end of the third portion forms a distal end of the first catheter, wherein the distal end of the third portion is disposed distal to the second catheter when the first catheter is advanced.

19. A method, comprising:
coupling a delivery device to a catheter assembly, wherein the catheter assembly comprises:
a catheter adapter, comprising a distal end, a proximal end, a lumen extending between the distal end and the proximal end; and
a second catheter secured to the catheter adapter and extending distally from the catheter adapter, wherein the transition portion is disposed within the catheter adapter when the first catheter is advanced, wherein the delivery device comprises:
a housing, comprising a distal end, a proximal end, and a slot;
a first catheter, comprising a proximal end and a distal end, wherein the first catheter comprises a first portion, a second portion, and a transition portion disposed between the first portion and the second portion, wherein the first portion includes a first inner diameter along a length of the first portion and a first outer diameter along the length of the first portion, wherein the second portion is disposed distal to the first portion, wherein the second portion includes a second inner diameter and a second outer diameter along a length of the second portion, wherein the second inner diameter is less than the first inner diameter, wherein the second outer diameter is less than the first outer diameter; and
an advancement tab moveable along the slot to advance the first catheter in a distal direction, wherein the transition portion is disposed distal to the distal end of the housing when the first catheter is advanced; and
advancing the first catheter in a distal direction, wherein in response to advancing the first catheter in the distal direction, the transition portion is disposed within the catheter adapter.

20. The method of claim 19, wherein the first catheter further comprises a third portion and another transition portion disposed distal to the first portion and the second portion, wherein the third portion includes a third inner diameter along a length of the third portion and a third outer diameter along a length of the third portion, wherein the third inner diameter is less than the second inner diameter, wherein the third outer diameter is less than the second outer diameter, wherein the other transition portion is disposed within the catheter when the first catheter is advanced.
